(19) United States
(12) Patent Application Publication
(10) Pub. No.: US 2012/0207839 A1
(43) Pub. Date: Aug. 16, 2012

(54) Mineralized Collagen/Bioceramic Composite and Manufacturing Method Thereof

(75) Inventors: SUNG-TSUEN LIU, Miaoli County (TW); SUNG-CHING CHEN, New Taipei City (TW); WAN-CHING HSU, New Taipei City (TW)

(73) Assignee: MAXIGEN BIOTECH INC., New Taipei City (TW)

(21) Appl. No.: 13/026,729

(22) Filed: Feb. 14, 2011

(51) Int. Cl. A61K 9/14; B29B 9/08; B29C 35/16; A61K 33/42; A61P 19/00

(52) U.S. Cl. 424/489; 424/602; 264/28; 264/5

(57) ABSTRACT

The present invention discloses a mineralized collagen/bioceramic composite useful as a hard tissue replacement material or substitute material, comprising about 10% to 95% by weight of mineralized collagen and about 5% to 90% by weight of bioceramics, and a method of manufacturing the same. Wherein, the mineralized collagen is used as a binder for the bioceramics, such as calcium phosphate ceramics, calcium sulfate ceramics, calcium carbonate ceramics, and other biocompatible ceramics. The bioceramic used in the mineralized collagen/bioceramic composite can be either in powder form or in granular form.

### BACKGROUND OF THE INVENTION

1. Field of the Invention

The present invention relates to a composite and its manufacturing method useful in orthopedic and maxillofacial surgeries and dental applications, and more particularly to a mineralized collagen/bioceramic composite useful as a hard tissue replacement material or substitute material and a manufacturing method thereof.

2. Description of Related Art

The composition of hard tissue, such as natural bone, comprises collagen and inorganic calcium phosphate, particularly biological apatite. Bone contains about 60 to 75% by weight of biological apatite and tooth has more than 98% by weight of biological apatite. Biological apatite is a naturally occurring calcium apatite-type material which is formed in the body by precipitation from body fluid at body conditions. This biological apatite has a structure which is similar to pure hydroxyapatite (HA), but contains some substitute ions for the calcium, phosphate and hydroxyl ions. Strictly speaking, synthetically produced precipitated HA is more similarly to biological apatite than are the HA ceramics. However, the precipitated HA has very fine particle size. Because of manipulation requirement, this hinders the applications of precipitated HA in medical area.

In the last twenty five years or so, many types of calcium phosphate ceramics have been prepared. Among these, HA, β-tricalcium phosphate (β-TCP), biphasic calcium phosphate (BCP) and calcium phosphate-containing glass have been extensively studied. Clinical studies confirmed that most of the calcium phosphate ceramics have excellent biocompatibility and are well accepted by both hard and soft tissue. The experimental result also indicated that dense HA is non-bioresorbable while other porous calcium phosphate ceramics are bioresorbable. Calcium phosphate ceramics have been approved as useful and biocompatible materials for bone substitutes. These include dicalcium phosphate dihydrate (DCPD), tricalcium phosphate (TCP), apatite compounds and tetracalcium phosphate (TTCP). Most of the calcium phosphate ceramics for medical application are prepared either as granular form or block form. The granular form has a mobility problem while the block form is very brittle and is difficult to shape. In order to solve the problems, many attempts have been made to prepare bioresorbable grouts or cementing material. Among these are Plaster of Paris, collagen and several types of calcium phosphate cement. The calcium phosphate cements developed can be classified as HA cement and DCPD cements. Plaster of Paris is resorbed too fast to match the bone growth. Similar to HA ceramic, HA cement is resorbed too slowly. On the other hand, dicalcium phosphate is too acidic and very difficult to control the setting composition and the resorption rate.

Collagen is a natural polymer and is the major component of skin and is also the major organic component of bone. In fact, bone is formed from mineralized collagen. In principle, mineralized collagen, particularly the HA mineralized collagen, should be an ideal material for bone implant material. Recently many studies have been devoted to prepare synthesized mineralized collagen. U.S. Pat. Nos. 5,455,231 and 5,231,169 and foreign patent WO 93/12736 to Brent R. Constantz et al. describe methods of mineralizing collagen by dispersing collagen in an alkaline solution and subsequently mixing calcium- and phosphate-containing solutions to the collagen for over an hour while maintaining the resulting collagen slurry at a pH of 10 or higher. In U.S. Pat. No. 5,320,844, Liu teaches the mineralization of collagen by strong mixing a calcium-containing solution and a phosphate-containing solution in collagen slurry at pH value at least 7 or preferable near 10 or higher. In U.S. Pat. Nos. 6,300,315 and 6,417,166, Liu further discloses the method of preparation of mineralized collagen membrane. In the U.S. Pat. Nos. 6,384,197 and 6,384,196, Wels et al. discuss the process for the formation of mineralized collagen fibrils, where the fibril formation and mineralization take place in one step. Several other studies (U.S. patent No. 2005/0217538, U.S. Pat. Nos. 6,902,584, 6,764,517, and 6,187,047) involve the formation of porous mineralized collagen with soluble binder which is rendered insoluble by cross-linking. The above studies use soluble collagen for the mineralization substance. Other mineralization techniques involve the mineralization of insoluble collagen fiber by double diffusion of calcium-containing solution and phosphate-containing solution into the reactor containing insoluble collage fiber or membrane. These include U.S. patent No. 2006/0204581 to Gower et al., U.S. Pat. No. 6,589,590 to Crermuszka et al. and U.S. Pat. No. 5,532,217 to Silver et al. Still other mineralization of collagen is prepared by using HA precursor and collagen. Many clinical studies confirmed the excellent biocompatibility and bioresorption character of the mineralized collagen materials.

Previously, in U.S. Pat. No. 5,425,770, Piez and his co-worker suggested the used of physical mixture of calcium phosphate ceramic with atelopeptide collagen composite material for bone repair. The collagen is served as binder for calcium phosphate ceramics. Collagen used ranges from 9% to 13% and calcium phosphate ceramics used covers from 87% to 91%. However, none of the previous works have disclosed that mineralized collagen can be applied as binder for the bioceramic system. Several clinical studies reported that mineralized collagen is a useful hard tissue implant material. It provides excellent tissue response. Besides, mineralized collagen also shows some superior physical properties than the pure collagen. This improvement in physical properties includes the increase of mechanical strength and more resistance to water degradation. For hard tissue implant material, besides the biocompatibility, both the mechanical strength and bioresorption rate are of important properties of application. Therefore, the present invention is aimed to provide the new mineralized collagen/bioceramic composite with the flexibility in controlling the swelling ratio, bioresorption rate, and mechanical strength.

### SUMMARY OF THE INVENTION

Accordingly, an object of the present invention is to provide a mineralized collagen/bioceramic composite and a manufacturing method thereof having excellent biocompatibility and controllable volume swelling ratios, bioresorption rates, and mechanical strength and being useful for bone grafts, bone substitutes and bone fillers.

According to the object of the present invention, it is provided with a mineralized collagen/bioceramic composite, comprising about 10% to 95% by weight of mineralized collagen and about 5% to 90% by weight of bioceramics, wherein the mineralized collagen serves as a binder for the bioceramics.

Preferably, the mineralized collagen comprises a substantially homogeneous mineralized collagen composite consisting essentially of about 25% to 95% by weight of collagen and about 5% to 75% by weight of calcium phosphate minerals precipitated from a collagen slurry by a soluble calcium ion-containing solution and a soluble phosphate ion-containing solution.

Preferably, the bioceramics selected in the mineralized collagen/bioceramic composite include calcium phosphate ceramics, calcium sulfate ceramics, calcium carbonate ceramics, and a combination thereof.

Preferably, the composite materials may be in the form of sheet, film, membrane, cylinder, block or granule.

Preferably, the mineralized collagen/bioceramic composite further comprising a drug selected from a group consisting of antibiotics, bone morphogenetic proteins, bone growth factors, skin grow factors, anti-scarring agents, and a combination thereof.

Furthermore, the present invention further provides a manufacturing method of a mineralized collagen/bioceramic composite, comprised the following steps: providing a mineralized collagen slurry; mixing the mineralized collagen slurry with bioceramics to form a mixture slurry; molding the mixture slurry into a desired shape; and drying or freeze-drying the mixture slurry to obtain a mineralized collagen/bioceramic composite.

Preferably, the manufacturing method further comprises a step of using a crosslinking reagent to crosslink with the mineralized collagen slurry or the mineralized collagen/bioceramic composite.

Briefly, the mineralized collagen/bioceramic composite and the manufacturing method thereof according to the present invention can provide one or more advantages as follows. The bioresorption rate and mechanical strength of the mineralized collagen/bioceramic composite can be easily manipulated by, for example, changing the mineralized collagen compositions, the types, particle sizes and amounts of the bioceramics, and types of solid forms. That is, the present invention can control the bioresorption rates and mechanical strength of the mineralized collagen/bioceramic composite depending on portions and areas of hard tissue to be repaired. Therefore, the mineralized collagen/bioceramic composite of the present invention gives flexibility in controlling the bioresorption rate for medical use and provide reasonable good mechanical strength. Besides, the mineralized collagen/bioceramic composite shows nice integrity even after weeks of aging in water.

Other aspects of the present invention will be illustrated partially in the subsequent detailed descriptions, conveniently considered partially through the teachings thereof, or comprehended by means of the disclosed embodiments of the present invention. Various aspects of the present invention can be understood and accomplished by using the components and combinations specifically pointed out in the following claims. It is noted that the aforementioned summary and the following detailed descriptions of the present invention are exemplary and illustrative, rather than being used to limit the scope of the present invention thereto.

### BRIEF DESCRIPTION OF THE DRAWINGS

The exemplary embodiments of the present invention will be understood more fully from the detailed description given below and from the accompanying drawings of various embodiments of the invention.

FIG. 1 illustrates a flow chart of a manufacturing method of a mineralized collagen/bioceramic composite in accordance with an embodiment of the present invention; and

FIG. 2 illustrates structure images of a mineralized collagen/bioceramic composite after aging in water for three weeks in accordance with another embodiment of the present invention.

### DETAILED DESCRIPTION OF THE PREFERRED EMBODIMENTS

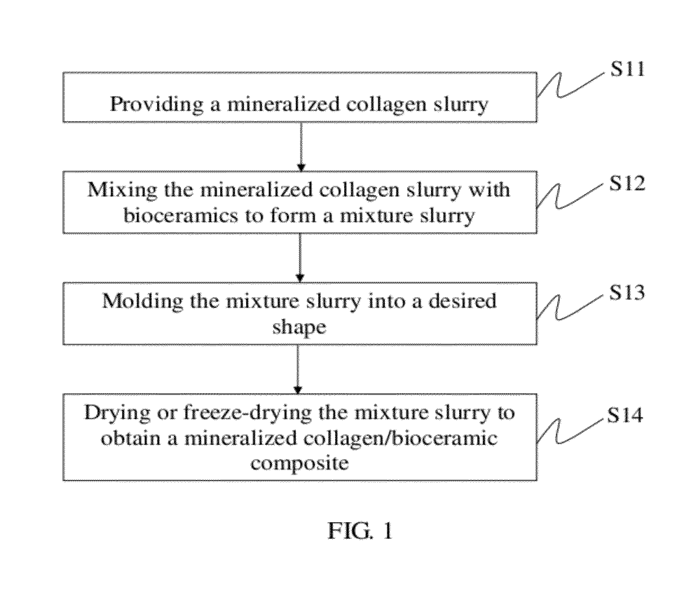

Referring to FIG. 1, which is a flow chart of a manufacturing method of a mineralized collagen/bioceramic composite in accordance with a first embodiment of the present invention. The method comprises the following steps. In step S11, a mineralized collagen slurry is provided. In step S12, the mineralized collagen slurry is mixed with bioceramics to form a mixture slurry. In step S13, the mixture slurry is casted and molded into a desired shape, and in step S14, the mixture slurry is dried or freeze-dried to obtain a mineralized collagen/bioceramic composite. The manufacturing method may further comprise a step of crushing, sieving and collecting the mineralized collagen/bioceramic composite in a granular form after the step S14.

At an embodiment, a method of making or producing a homogeneous mineralized collagen slurry comprises the steps of forming a collagen slurry, a soluble calcium ion-containing solution, and a soluble phosphate ion-containing solution, and adding the soluble calcium ion-containing solution and soluble phosphate ion-containing solution to the collagen slurry while stirring, preferably vigorously stirring, the collagen slurry and maintaining the pH value of said collagen slurry at least about 7, preferable near 10 or higher. At another embodiment, the method of preparing the mineralized collagen slurry may further comprise the following steps after the adding step: recovering the mineralized collage slurry by a solid-liquid separation method; and washing and recovering the mineralized collagen slurry with water to get the purified mineralized collagen slurry.

At other embodiments, a mineralized collagen/bioceramic composite of the present invention comprises about 10% to 95% by weight of mineralized collagen and about 5% to 90% by weight of bioceramics. The mineralized collagen may be a substantially homogeneous mineralized collagen and is used as a binder for the bio ceramics. The mineralized collagen may consist essentially of between about 25% and about 95% by weight of collagen and about 5% to 75% by weight of calcium phosphate minerals. The calcium phosphate minerals may be calcium phosphate, tricalcium phosphate (TCP), octacalcium phosphate (OCP), amorphous calcium phosphate (ACP), HA, apatite-like minerals, substitute apatite, calcium-deficient apatite (CDA), and a combination thereof.

Moreover, the bioceramics used in the preparation of the mineralized collagen/bioceramic composite may be calcium phosphate ceramics, calcium sulfate ceramics, calcium carbonate ceramics or their mixtures. Suitable calcium phosphate ceramics may be dicalium phosphate ceramics including dihydrate and anhydrous, TCP ceramics including α-TCP and β-TCP, tetracalcium phosphate (TTCP) ceramic, OCP ceramic, calcium pyrophosphate, hydroxyapatite (HA), carbonate apatite, fluoride apatite, apatite-type ceramic, apatite-like minerals, substitute apatite, CDA, and calcium alkaline phosphate such as CaNaPO₄ and CaKPO₄, and a combination thereof. Suitable calcium sulfate ceramics may be calcium sulfate dihydrate, calcium sulfate hemihydrate, calcium sulfate anhydrous, and a combination thereof. Calcium carbonate ceramics can be natural mineral, such as coral, or synthetic material. The mineralized collagen/bioceramic composite may be in sheet form, membrane form, cylinder form, block form, or granule form.

For preparation of the mineralized collagen/bioceramic composite of the present invention, any suitable collagen component, including natural collagen or recombinant collagen, may be used. The natural collagen sources may come from skin, tendon, or bone of animal such as bovine, porcine, equine, chicken, or the like. The preferred starting collagen materials are non-mineralized collagen. The initial collagen material can be any solid form, solution or slurry.

The initial step in the preparation of the mineralized collagen/bioceramic composites may be the preparation of collagen slurry. If the solid collagen is used, it is preferably dispersed in an acid or alkaline solution to form homogeneous gel-type slurry. The concentration of the collagen slurry suitable for following mineralization processes is preferably between about 0.1% and about 5%.

In general, either a soluble calcium ion-containing solution (for example, a soluble calcium salt) or a soluble phosphate ion-containing component (for example, a soluble phosphate salt) is then dissolved or otherwise directly combined into the collagen slurry. If a calcium ion-containing component is directly combined into the collagen slurry, the second, phosphate ion-containing component is preferably separately dissolved or otherwise combined in a liquid medium, preferably water, to form a solution. In either such case, the second (phosphate ion-containing or calcium ion-containing) component is preferably quick added (for example, by pouring) into the collagen slurry.

Alternately, two separate solutions, one with a soluble calcium ion-containing component and the other with phosphate ion-containing components, can be prepared, and the two solutions are preferably quickly and simultaneously added (poured) into the collagen slurry or the two solutions may be added to the collagen slurry slowly. Preferably, but not necessarily, stoichiometric amounts of calcium and phosphate ions are added to the collagen slurry.

In either case, during the combination step, the collagen slurry is vigorously mixed or stirred to ensure the formation of homogeneous slurry reaction product. Although the rapidity of adding the calcium ion-containing component or phosphate ion-containing component, or both, to the collagen slurry is not critical, the addition is preferably quickly performed to ensure homogeneous reaction product. After the complete addition of the calcium ion-containing and phosphate ion-containing component to the collagen slurry, the slurry is either continuously stirred or allowed to stand un-stirred until the precipitation of calcium phosphate is completed.

During the preparation procedure, the temperature of the mixture is preferably maintained below about 40° C. Moreover, during the precipitation of calcium phosphate, the collagen slurry is preferably maintained at pH value of at least 7.0 and preferably at a pH value of at least 9.0. This pH control can be achieved by adding enough alkaline solution, such as sodium hydroxide, potassium hydroxide or ammonium hydroxide, to either the collagen slurry or phosphate ion-containing solution or calcium ion-containing solution before its combining with the slurry.

A calcium phosphate saturated solution at a pH value near 8 or higher will normally induce the precipitation of HA, substitute apatite or calcium apatite-like calcium phosphate minerals. Other components may also be incorporated into the calcium phosphate mineral. For example, if carbonate apatite or fluoride apatite is to be incorporated into the mineralized collagen product, a soluble carbonate or soluble fluoride salt can be added into the phosphate ion-containing solution before its addition to the collagen slurry. The calcium phosphate mineral deposited in the collagen slurry at a slurry pH value near neutral or up to 8 is most likely calcium phosphate, TCP, OCP, ACP, HA, CDA, substitute apatite, apatite-like minerals, or a combination thereof. At a slurry pH of about 8 or higher, the most probable precipitation product is HA or calcium apatite-like minerals. In order to induce the precipitation of calcium apatite materials in the collagen slurry, the preferred mole ratio of calcium to phosphate in the initial solutions is about 1 to 2, and is more preferably about 1.67. However, other mole ratio can also be used.

After the calcium phosphate mineral is completely precipitated, the resulting mineralized collagen slurry is separated and purified, for example by being filtered and/or centrifuged and/or washing several times until the material is free of other soluble components, such as entrapped soluble impurities. In the mineralized collagen, the calcium phosphate-containing component (i.e. calcium phosphate mineral) is deposited on both surface and inside of the collagen fiber. The purified mineralized collagen is then collected.

The bioceramic in fine powder form having particle sizes from few microns to about 100 μm, or in granular form having particle sizes from about 0.1 mm to about 5 mm is then added to the purified mineralized collagen slurry. The mixtures are then mixed to form the mineralized collagen/bioceramic composite of the present invention.

A drug or a combination of drugs may be incorporated into the mineralized collagen/bioceramic composite by adding the drug or drugs into the mineralized collagen before further processing the slurry into final products. The drug or drugs may include antibiotics, bone morphogenetic proteins, other bone growth factors, skin growth factors, anti-scarring agents and/or combinations thereof. In such case, the drugs are added with bioceramics to the purified mineralized collagen slurry before processing to the final products.

In the processing of the mineralized collagen/bioceramic composite, after the addition of bioceramics and/or the addition of drugs to the purified mineralized collagen slurry, the composite mixture may be then casted, shaped or molded to the desired shape of sheet form, membrane form, block form or cylinder form. After that, the composite mixture is then air dried or freeze-dried. The composite material can then be further processed as granular form. Suitable granular form of medical application will be in the size from 0.1 mm to about 5 mm.

In order to enhance the mechanical strength of the mineralized collagen/bioceramic composite material, a collagen crosslinking reagent can be added into the mineralized collagen slurry after the precipitation and before the purification steps described above. As an alternative, the dried mineralized collagen/bioceramic composite may be soaked in the collagen crosslinking agent. After the crosslinking process is completed, the composite material is then soaked and washed with pure water to remove any unreacted crosslinking agent.

Another method to enhance the mineralized collagen/bioceramic composite is repeatedly coating the composite with collagen or mineralized collagen. In this process, the dry product of mineralized collagen/bioceramic composite is repeatedly coated with the mineralized collagen slurry or pure collagen slurry and dried.

It is apparent that the present mineralized collagen/bioceramic composite is quite different from a pure collagen/bioceramic composite. Pure collagen/bioceramic composites are quite weak when soaked in water and show high degree of swelling. Furthermore, pure collagen/bioceramic composites are difficult to handle and their bioresorption rate is difficult to control. However, the mineralized collagen/bioceramic composite shows nice integrity even after weeks of aging in water. Further, the bioresorption rate of the new mineralized collagen/bioceramic composite can be controlled by changing the content of calcium phosphate-containing minerals in the mineralized collagen, or by changing the type, particle size and amount of bioceramics used. In general, the decrease of the content of calcium phosphate-containing mineral in the mineralized collagen will increase the bioresorption. In the mineralized collagen/bioceramic composite, the use of calcium sulfate, calcium carbonate and dicalcium phosphate will show faster bioresorption rate than those of other calcium phosphate ceramics such as HA or TCP.

### EXAMPLES

### Example 1

Preparation of Mineralized Collagen Slurry: 1 g of solid fibril collagen (type I collagen) is added into a container with 250 ml of pure water. 5.3 g of Na₃PO₄.12H₂O is added into the water. The aqueous mixture is then stirred (mixed) in a blender until the collagen is in the form of homogeneous gel slurry. The pH value of the collagen is higher than 10.

3.54 g of Ca(NO₃)₂.4H₂O is dissolved in 50 ml of pure water to form a calcium nitrate solution. The collagen slurry is kept in the blender and stirred vigorously when the Ca(NO₃)₂ solution is poured into the collagen slurry. The stirring is continued for several more minutes and then kept unstirred for one hour. The final pH value of the collagen slurry is still maintained near 10 or higher after the reaction. The slurry is then filtered with a separation funnel and washed several times with pure water until it is free of soluble impurities. If HA is the calcium phosphate deposited in the collagen and no weight lost during the process, The mineralized collagen slurry should contains 1 g collagen and 1.5 g precipitated HA (40% collagen and 60% precipitated HA in the mineralized collagen).

One fourth of the above purified mineralized collagen slurry is shaped into a rectangular shape. The mineralized collagen is then air dried at room temperature. The weight of the air dried sample is about 0.6 g. This air dried sample does not show significant swelling and keeps integrity after aging in water.

### Example 1-1

One half of the above purified mineralized collagen slurry is mixed with 5 g of HA granule with a particle size between 0.5 mm and 2 mm. The mixed mineralized collagen is then shaped into a rectangular shape and air dried at room temperature. The dried mineralized collagen/HA ceramic composite has weight 6.25 g (1.25 g mineralized collagen and 5 g HA granule, i.e. 20% mineralized collagen and 80% HA). This composite material stays strong and does not show sign of disintegration after aging in water for several weeks.

### Example 2

Preparation of Mineralized Collagen Slurry: 0.5 g of solid fibril collagen (type I collagen) is added into a container with 100 ml of pure water. 5.0 g of Na₃PO₄.12H₂O is added into the water. The aqueous mixture is then stirred (mixed) in a blender until the collagen is in the form of homogeneous gel slurry. The pH value of the collagen is higher than 10.

2.53 g of Ca(NO₃)₂.4H₂O is dissolved in 50 ml of pure water to form a calcium nitrate solution. The collagen slurry is kept in the blender and stirred vigorously when the Ca(NO₃)₂ solution is poured into the collagen slurry. The stirring is continued for several more minutes and then kept unstirred for one hour. The final pH of the collagen slurry is still maintained near 10 or higher after the reaction. The slurry is then filtered with a separation funnel and washed several times with pure water until it is free of soluble impurities. If HA is the calcium phosphate deposited in the collagen and no weight lost during the process, the mineralized collagen slurry should contains 0.5 g collagen and 1.07 g precipitated HA (31.8% collagen and 68.2% precipitated HA in the mineralized collagen).

### Example 2-1

One fourth of the purified mineralized collagen slurry prepared from Example 2 is mixed with 2 g of dicalcium phosphate dihydrate (CaHPO₄.2H₂O) granule with a particle size from 1 mm to 2 mm. The mixture of slurry is the shaped into a rectangular shape and air dried in room temperature. The dried mineralized collagen/CaHPO₄.2H₂O composite contains 16.7% mineralized collagen and 83.3% dicalcium phosphate dihydrate ceramic. The dry composite shows some elasticity and is not as rigid as regular ceramic material. This mineralized collagen/CaHPO₄.2H₂O composite keeps good integrity when aged in water.

### Example 2-2

One fourth of the purified mineralized collagen slurry prepared from Example 2 is mixed with 1 g fine powder of calcium sulfate anhydrous (CaSO₄). The slurry mixture is then molded into a block form and air dried. The dried composite is then further processes into granular form having the particle size of 0.5 to 3 mm. This mineralized collagen/CaSO₄ composite material contains 28% mineralized collagen and 72% CaSO₄ ceramic.

### Example 2-3

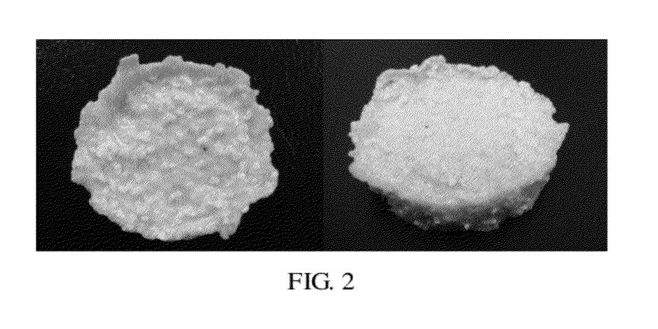

The above purified mineralized collagen slurry prepared from Example 2 is mixed with bioceramic granules. The dried mineralized collagen/bioceramic composite is composed of 50 wt % mineralized collagen and 50 wt % bioceramic (60 wt % HA and 40 wt % β-TCP). The particle sizes of the bioceramic granules are in range of 0.5 mm to 2 mm. The composite material stays strong and does not show sign of disintegration after aging in water for three weeks, as shown in FIG. 2.

### Example 2-4

The above purified mineralized collagen slurry prepared from Example 2 is mixed with bioceramic granules in different ratios. The particle sizes of the bioceramic granules are in range of 0.5 mm to 2 mm. Two kinds of dried mineralized collagen/bioceramic composites, respectively composed of 20 wt % mineralized collagen and 75 wt % bioceramic (100 wt % HA), and 50 wt % mineralized collagen and 50 wt % bioceramic (100 wt % HA), are tested on their volume swelling ratios and compressive moduli, compared to 100 wt % mineralized collagen. The results are shown as follows. Volume swelling ratio (%)=(Volume of sample after immersing in water−Volume of sample before immersing in water)/(Volume of sample before immersing in water)×100%. Therefore, the present invention can provide the mineralized collagen/bioceramic composite material with the flexibility in controlling the swelling ratio and the mechanical strength by adjusting the ratio of the mineralized collagen to bioceramics and/or the kinds of bioceramics.

| | Properties | | | |
|---|---|---|---|---|
| | Volume Swelling Ratio (%) | | | Compressive |
| Sample | 24 hrs | 48 hrs | 72 hrs | Modulus (MPa) |
| 100 wt % Mineralized | 71 | 79 | 89 | 0.88 |
| Collagen | | | | |
| 50 wt % Mineralized | 61 | 63 | 65 | 0.65 |
| Collagen and 50 wt % | | | | |
| Bioceramic | | | | |
| 25 wt % Mineralized | 47 | 48 | 50 | 0.48 |
| Collagen and 75 wt % | | | | |
| Bioceramic | | | | |

While this has been described with respect to various specific examples and embodiments and method of making the mineralized collagen/bioceramic composite material, it is to be understood that the invention not limited thereto. Consequently, any and all variation and/or equivalent methods which may occur to that skill in the applicable art are to be considered to be within the scope and spirit of the invention as set forth in the claims which are appended hereto as part of this application.

What is claimed is:

1. A mineralized collagen/bioceramic composite, comprising about 10% to 95% by weight of mineralized collagen and about 5% to 90% by weight of bioceramics, wherein the mineralized collagen is used as a binder for the bioceramics.

2. The mineralized collagen/bio ceramic composite according to claim 1, wherein the mineralized collagen comprises a substantially homogeneous mineralized collagen composite consisting essentially of about 25% to 95% by weight of collagen and about 5% to 75% by weight of calcium phosphate minerals precipitated from a collagen slurry by a soluble calcium ion-containing solution and a soluble phosphate ion-containing solution.

3. The mineralized collagen/bioceramic composite according to claim 2, wherein the collagen is natural collagen, recombined collagen or a combination thereof.

4. The mineralized collagen/bioceramic composite according to claim 2, wherein the calcium phosphate minerals are selected from a group consisting of calcium phosphate, tricalcium phosphate, octacalcium phosphate, hydroxyapatite, apatite-like minerals, substitute apatite, calcium-deficient apatite, and a combination thereof.

5. The mineralized collagen/bioceramic composite according to claim 1, wherein the bioceramics are selected from a group consisting of calcium phosphate ceramics, calcium sulfate ceramics, calcium carbonate ceramics, and a combination thereof.

6. The mineralized collagen/bioceramic composite according to claim 5, wherein the calcium phosphate ceramics have a mole ratio of calcium to phosphate ranging from 1.0 to near 2.

7. The mineralized collagen/bioceramic composite according to claim 5, wherein the calcium phosphate ceramics are selected from a group consisting of dicalcium phosphate dihydrate, dicalcium phosphate anhydrous, α- and β-tricalcium phosphate, tetracalcium phosphate, octacalcium phosphate, calcium pyrophosphate, hydroxyapatite, apatite-like minerals, substitute apatite, calcium-deficient apatite, and a combination thereof.

8. The mineralized collagen/bioceramic composite according to claim 5, wherein the calcium sulfate ceramics are selected from a group consisting of calcium sulfate dihydrate, calcium sulfate hemihydrate, calcium sulfate anhydrous, and a combination thereof.

9. The mineralized collagen/bioceramic composite according to claim 5, wherein the calcium carbonate ceramics are selected from a group consisting of synthetic calcium carbonate, natural minerals, and a combination thereof.

10. The mineralized collagen/bioceramic composite according to claim 1, wherein the mineralized collagen is non-crosslinked.

11. The mineralized collagen/bioceramic composite according to claim 1, wherein the mineralized collagen is crosslinked.

12. The mineralized collagen/bioceramic composite according to claim 1, wherein the bioceramics are in granular form with a particle size ranging from about 0.1 mm to about 5 mm, or in powder form with 100 μm or less of the particle size, or a combination thereof.

13. The mineralized collagen/bioceramic composite according to claim 1, comprising a sheet form, membrane form, cylinder form, block form, or granule form.

14. The mineralized collagen/bioceramic composite according to claim 1, further comprising a drug selected from a group consisting of antibiotics, bone morphogenetic proteins, bone growth factors, skin grow factors, anti-scarring agents, and a combination thereof.

15. A manufacturing method of a mineralized collagen/bioceramic composite, comprising steps of:
providing a mineralized collagen slurry;
mixing the mineralized collagen slurry with bioceramics to form a mixture slurry;
molding the mixture slurry into a desired shape; and
drying or freeze-drying the mixture slurry to obtain a mineralized collagen/bioceramic composite.

16. The manufacturing method of claim 15, further comprising a step of crushing, sieving and collecting the mineralized collagen/bioceramic composite in a granular form after the drying or freeze-drying step.

17. The manufacturing method of claim 15, further comprising a step of repeatedly coating the mineralized collagen/bioceramic composite with the mineralized collagen slurry or pure collagen slurry after the drying or freeze-drying step.

18. The manufacturing method of claim 15, further comprising a step of using a crosslinking reagent to crosslink with the mineralized collagen slurry or the mineralized collagen/bioceramic composite.

19. The manufacturing method of claim 15, wherein the mineralized collagen slurry is prepared by a method comprising steps of:
providing a collagen slurry, a soluble calcium ion-containing solution, and a soluble phosphate ion-containing solution; and
adding the soluble calcium ion-containing solution and the soluble phosphate ion-containing solution to the collagen slurry while stirring the collagen slurry with maintaining a pH value at least about 7 or higher, thereby inducing precipitation of calcium phosphate minerals in the collagen as the mineralized collagen slurry.

20. The manufacturing method of claim 19, wherein the method of preparing the mineralized collagen slurry further comprises the following steps after the adding step:
recovering the mineralized collage slurry by a solid-liquid separation method; and
washing and recovering the mineralized collagen slurry with water to get the purified mineralized collagen slurry.
